(19) United States
(12) Patent Application Publication
(10) Pub. No.: US 2006/0111765 A1
(43) Pub. Date: May 25, 2006

(54) Medical device for thermally affecting tissue having an inflatable circumferential stiffening member

(75) Inventor: John M. Kirkman JR., Kingston, NH (US)

Correspondence Address: John Christopher;Christopher & Weisberg, P.A., Suite 2040, 200 East Las Olas Boulevard, Fort Lauderdale, FL, 33301, US

(73) Assignee: Seacoast Technologies, Inc.

(21) Appl. No.: 10/993,332

(22) Filed: Nov. 19, 2004

(51) Int. Cl. A61F 7/00; A61F 7/12

(52) U.S. Cl. 607/104; 607/105; 607/113

(57) ABSTRACT

A medical device for thermally affecting tissue including a heat exchanger having a first fluid circulating through the heat exchanger, a deployment member secured to at least a portion of the heat exchanger, and a second fluid located within the deployment member. The medical device may be deformable upon contact with tissue, and may further include a deployment member having a rigidity that can be modified by changes in temperature.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a method and system for thermally affecting tissue.

### BACKGROUND OF THE INVENTION

Researchers and physicians have long recognized the consequences of reduction of body temperature in mammals, including induction of stupor, tissue damage, and death. Application of freezing and near freezing temperatures to selected tissue is commonly employed to preserve tissue and cell (e.g. sperm banks); and application of extreme cold (far below freezing) is effective for tissue ablation. However, localized cooling (not freezing) of tissue has generally been limited to the placement of an “ice-pack” or a “cold compress” on injured or inflamed tissue to reduce swelling and the pain associated therewith. Localized cooling of internal organs, such as the brain, has remained in large part unexplored.

For example, “brain cooling” has been induced by cooling the blood supply to the brain for certain therapies. However, as the effects of the cool blood cannot be easily localized, there is a systemic temperature reduction throughout the body that can lead to cardiac arrhythmia, immune suppression and coagulopathies.

Although attempts have been made to localize cooling of the brain with wholly external devices, such as cooling helmets or neck collars, there are disadvantages associated with external cooling to affect internal tissue. For example, external methods do not provide adequate resolution for selective tissue cooling, and some of the same disadvantages that are associated with systemic cooling can occur when using external cooling devices. Further, internal cooling devices have also been developed, but are often limited in their ability to conform to the shapes of brain tissue targeted for cooling. Exemplary devices include catheters and inflatable balloons through which heated or cooled fluids are circulated. While it is known to use balloons to contact tissue surfaces along the length of a catheter that is inserted into a vessel, a need arises for a device to apply localized thermal energy in alternate treatment scenarios.

Moreover, it is also desirable to avoid creating unnatural openings in a human body. However, when a medical need mandates creating an opening, making as small an opening as possible is advantageous. The need to keep openings to a minimum is particularly applicable when dealing with openings in a human skull, yet a device is needed to apply or remove thermal energy to or from a tissue area with a larger surface area than the opening through which the catheter is inserted.

Further, problems of uniform thermal distribution arise with known thermal transfer devices. When a thermally transmissive fluid is infused into a space, the distribution of thermal energy is governed by the function of thermal convection. As such, in many situations thermal energy is not evenly distributed throughout the space.

In view of the above limitations, it would be desirable to provide a device which evenly distributes or removes thermal energy from tissue and is capable of thermally affecting large tissue areas while remaining implantable through a small opening in a patient.

### SUMMARY OF THE INVENTION

The present invention advantageously provides a medical device which evenly distributes or removes thermal energy from tissue and is capable of thermally affecting large tissue areas while remaining implantable through a small opening in a patient.

In an exemplary embodiment, the medical device includes a heat exchanger that defines a chamber adapted to receive a first fluid, which can circulate through the heat exchanger. The medical device further includes a deployment member secured to at least a portion of the heat exchanger, with the deployment member capable of receiving a second fluid. Either of the two fluids can be thermally transmissive fluids which are chilled to below body temperature.

The medical device can be constructed from pliant materials, thereby enabling the device to deform when in contact with tissue. Further, the pressure of the second fluid located within the deployment member can be modified for a desired rigidity of the deployment member. Moreover, the deployment member can be constructed from a material whose rigidity can be modified by changes in temperature, and can further be detachable from the heat exchanger.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 illustrates an exemplary cooling system used to perform a medical procedure in accordance with the present invention;

FIG. 2 depicts an embodiment of the present invention in both a minimized state and a deployed state;

FIG. 3 illustrates an embodiment of a cooling structure in accordance with the present invention;

FIG. 4 shows a cross-section of a medical device in accordance with the present invention;

FIG. 5 depicts a variation of a cross-section of a medical device in accordance with the present invention;

FIG. 6 illustrates a variation of an embodiment of a medical device in accordance with the present invention;

FIG. 7 illustrates an alternative variation of an embodiment of a medical device in accordance with the present invention;

FIG. 8 shows yet another alternative variation of an embodiment of a medical device in accordance with the present invention; and

FIG. 9 depicts an exemplary cooling system used to perform a medical procedure in accordance with the present invention.

### DETAILED DESCRIPTION OF THE INVENTION

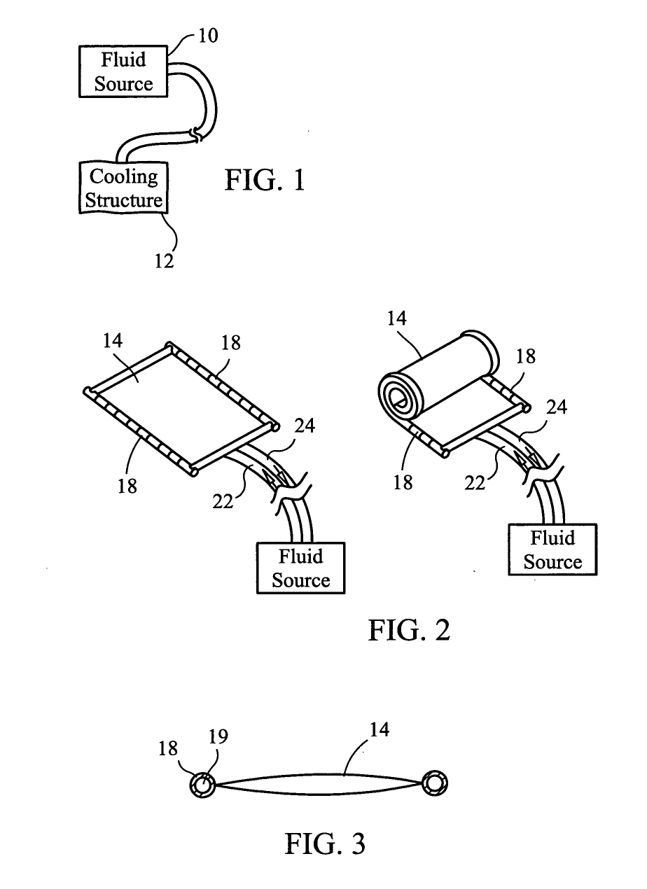

Referring to FIG. 1, the present invention provides a medical device for thermally affecting tissue, generally including one or more fluid sources 10 that are in fluid communication with a cooling structure 12.

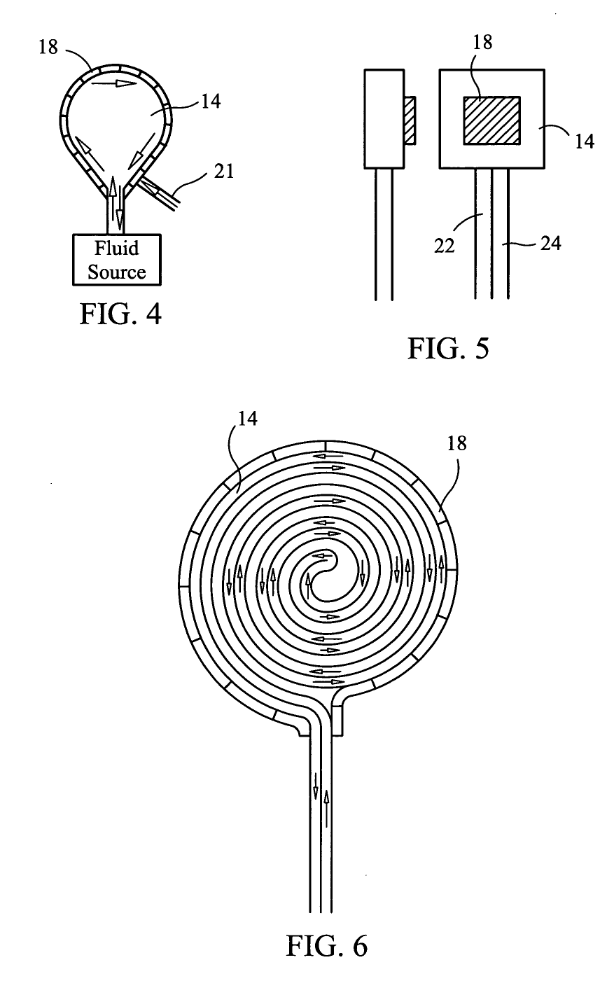

Particularly, as shown in FIG. 2, the present invention provides a medical device for thermally affecting tissue having a heat exchanger 14 that defines a chamber for receiving a first fluid so that it circulates through the heat exchanger 14. The medical device further includes a deployment member 18 which is secured to at least a portion of the heat exchanger 14. The deployment member 18 allows the medical device to eventually thermally affect a tissue area that is larger than the desired surgical opening by providing a mechanical means by which to enlarge or expand the structure of the medical device upon implantation, as the medical device likely is temporarily deformed prior to insertion into a surgical opening. The heat exchanger 14 can include an input lumen 22 as well as an output lumen 24 to both introduce as well as evacuate fluid, respectively, from the chamber of the heat exchanger 14. The input lumen 22, the chamber, and the output lumen 24 can form a specific circulation path for the first fluid 16 to travel through. Moreover, the deployment member 18 can further be adapted to receive a second fluid into a deployment member lumen 19, shown in FIG. 3, and can further include an inflation lumen 21 for introducing the second fluid into the deployment member 18, as shown in FIG. 4.

The heat exchanger 14 can be constructed from a pliant material, including various plastic or silicone elastomer materials, or any other such material that would allow the heat exchanger 14 to deform when pressure is affixed to it such as by placement in contact with tissue. The deformability of the heat exchanger 14 allows the medical device to conform to an uneven or irregular tissue surface, thereby enhancing the ability of the device to thermally affect the tissue. Additionally, the heat exchanger 14 can be constructed from thermally transmissive materials having properties that influence the thermal conductivity, and thus the resulting effectiveness of the heat exchanger in thermally affecting the tissue. The heat exchanger 14 can have an essentially circular shape, essentially rectangular shape, or can be constructed to mirror the shape of a tissue region that will be thermally affected by the medical device of the present invention.

The deployment member 18 provides an expansive element that ensures that the medical device expands to proper shape after being temporarily deformed to fit through a small surgical opening. The deployment of the medical device ensures that the heat exchanger 14 covers the maximum amount of surface area of the tissue to be thermally affected. Moreover, the deployment member 18 provides structural rigidity for the medical device while maintaining the pliability of the heat exchanger 14, thereby reducing the difficulty in placing the medical device into the desired position in contact with the tissue to be thermally affected. The deployment member 18 can be constructed from a thermally transmissive material, and can further be disposed on an interior or exterior perimeter of the heat exchanger 14 or any portion of a surface thereof. Moreover, the deployment member 18 can also be located within a portion of the chamber of the heat exchanger 14.

The deployment member 18 can have a shape that is substantially similar to the shape of the heat exchanger 14, as shown in FIG. 5. Alternatively, the deployment member 18 can take the form of a tube-like structure, a flattened-rectangular rib, or any alternative shape that provides the described structural and deployment characteristics.

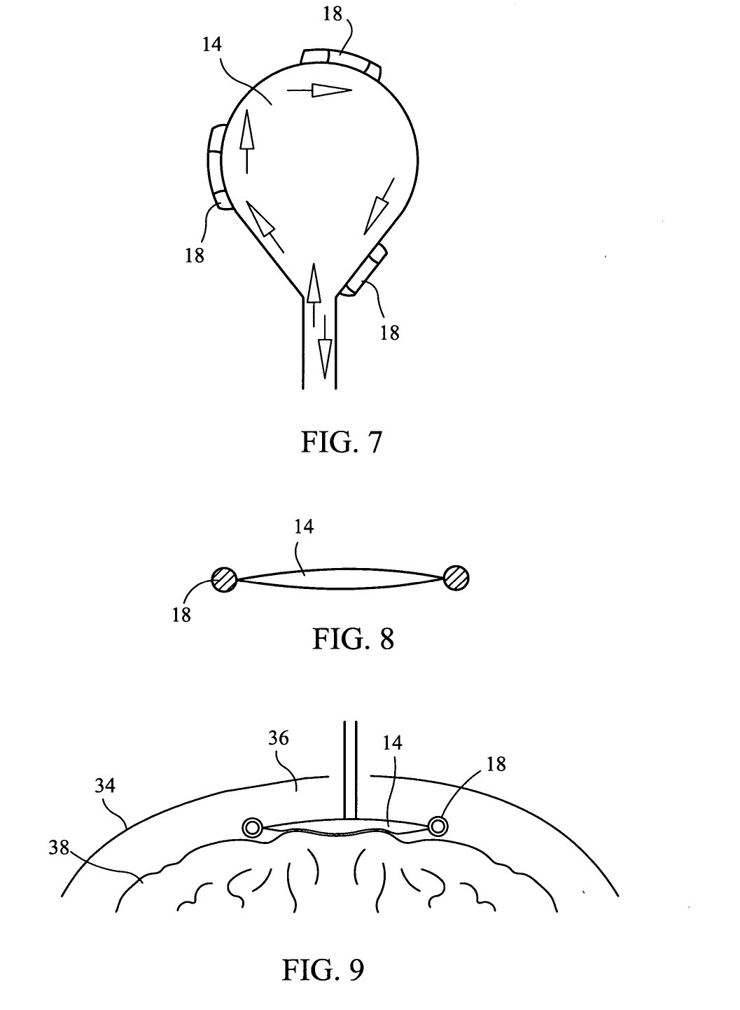

In an exemplary embodiment, the deployment member 18 is a single inflatable element, as shown in FIG. 6. However, the deployment member 18 may include multiple inflatable elements, as shown in FIG. 7. Further, the deployment member 18 can be detachable from the heat exchanger 14.

The first fluid that circulates through the heat exchanger 14 can be a thermally transmissive fluid, and can further be chilled to below body temperature in order to enhance the thermal effectiveness of the medical device. In an exemplary application, the first fluid can be cooled to a temperature of 4° C. to 37° C. Furthermore, the first fluid can be pressurized up to 1.0 psig, however, to maximize the pliability of the heat exchanger, the first fluid is preferably kept as low as possible while maintaining flow through the circulation path of the heat exchanger 14, as well as achieving a desired thermal result.

The second fluid that can be located in the deployment member 18 can be a thermally transmissive fluid. The second fluid can be pressurized to a predetermined pressure, wherein the predetermined pressure corresponds to a desired rigidity of the deployment member 18. The rigidity of the deployment member 18 can be modified to attain a desired level of expansion for the heat exchanger 14, as well as achieve differing levels of manipulability in order to ease the placement of the medical device into thermal contact with the tissue.

The circulation path can be configured such that the first fluid flows directly into and out of the chamber of the heat exchanger 14. Additionally, at least a portion of the circulation path can be looped around itself into a coil configuration, as shown in FIG. 6. However, the orientation of the circulation path is not limited to a particular geometric shape so long as the combination of the flow of the first fluid and the surface area of the heat exchanger 14 is sufficient to achieve a particular thermal result.

While an exemplary embodiment of the present invention includes introducing a fluid into the deployment member 18 for varying levels of rigidity, it is also beneficial to employ a deployment member 18 that does not include a fluid nor an inflation lumen for introducing a fluid, as shown by the cross-sectional illustration of FIG. 8. Rather, the deployment member 18 can be constructed from a material wherein the rigidity of the deployment member 18 can be modified by changes in temperature. The temperature-dependent material can be such that at room temperature, or at body temperature when implanted into the body, the deployment member is relatively pliant and flexible. Upon introducing a chilled first fluid into the chamber of the heat exchanger 14, the temperature of the deployment member 18 would reduce, and the rigidity of the deployment member can increase, thereby providing the desired expansion mechanism and ability to manipulate the heat exchanger into position.

Moreover, the deployment member 18 can be constructed from a shape memory material. Shape memory materials are generally known in the art, and have the ability to undergo structural alterations when exposed to various temperatures. The deployment member may additionally include a spring mechanism that slowly expands the heat exchanger when placed in thermal communication with the tissue.

Turning now to FIG. 9, a cooling structure is shown in use, wherein a portion of the skull 34 has been removed and the cooling structure placed in the space 36 between the surface of the brain 38 or its covering tissue, the dura, and the interior surface of the skull. Although not shown in the drawings, it is understood that in order to thermally affect tissue other than the brain, the medical device can be placed at other locations in or on a patient. For thermally affecting brain tissue, the skin and bone of a patient are penetrated, thus providing access for the implantation of the device. Techniques for such penetration and placement are generally known in the art. The medical device, prior to implantation, does not contain any fluid in either the heat exchanger 14 nor the deployment member 18, thereby allowing for the medical device to easily be implanted through an opening that is smaller than the area of the tissue to be affected. Upon implanting the medical device of the present invention in the general area to be thermally affected, the second fluid is introduced into the deployment member 18. This introduction of fluid results in the medical device having an increased degree of structural rigidity for more accurate placement of the medical device in thermal communication with the tissue, as well as expanding the heat exchanger 14 to its fully deployed shape. Once the medical device is positioned, the first fluid 16 is introduced into the heat exchanger 14. By introducing the first fluid at a relatively low pressure, the heat exchanger can maintain its pliability and thus conform to any uneven tissue surface, thereby maximizing its ability to thermally affect the tissue, while the deployment member 18 keeps the heat exchanger in a fully deployed state. When a particular thermal result is achieved, circulation of the first fluid can cease, and the first fluid can be evacuated from the first heat exchanger 14. Subsequently, the second fluid is evacuated from the deployment member 18, thereby easing the removal of the device from the patient worksite.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A medical device for thermally affecting tissue, comprising:
a heat exchanger defining a chamber adapted to receive a first fluid, and
a deployment member secured to at least a portion of the heat exchanger.

2. The medical device according to claim 1, wherein the deployment member is adapted to receive a second fluid.

3. The medical device according to claim 1, wherein the deployment member is disposed on an interior perimeter of the heat exchanger.

4. The medical device according to claim 1, wherein the deployment member is disposed on an exterior perimeter of the heat exchanger.

5. The medical device according to claim 1, wherein the deployment member traverses a portion of a surface of the heat exchanger.

6. The medical device according to claim 1, wherein the deployment member is located within the chamber of the heat exchanger.

7. The medical device according to claim 1, wherein the deployment member is shaped substantially similar to the heat exchanger.

8. The medical device according to claim 1, wherein the heat exchanger is made from a pliant material.

9. The medical device according to claim 1, wherein the heat exchanger is made from a thermally transmissive material.

10. The medical device according to claim 1, wherein the heat exchanger is deformable upon contact with tissue.

11. The medical device according to claim 1, wherein the heat exchanger has an essentially circular shape.

12. The medical device according to claim 1, wherein the heat exchanger has an essentially rectangular shape.

13. The medical device according to claim 1, wherein the heat exchanger further includes an input lumen and an output lumen.

14. The medical device according to claim 13, wherein the chamber, interior lumen, and output lumen define a fluid circulation path.

15. The medical device according to claim 1, wherein the first fluid is a thermally transmissive fluid.

16. The medical device according to claim 1, wherein the first fluid is pressurized to less than 1.0 psig.

17. The medical device according to claim 1, wherein the first fluid is chilled to below body temperature.

18. The medical device according to claim 2, wherein the second fluid is a thermally transmissive fluid.

19. The medical device according to claim 2, wherein the second fluid is pressurized to a predetermined pressure.

20. The medical device according to claim 19, wherein the predetermined pressure corresponds to a desired rigidity of the inflation member.

21. The medical device according to claim 1, wherein the deployment member is detachable from the heat exchanger.

22. The medical device according to claim 1, wherein the deployment member includes an inflation lumen.

23. The medical device according to claim 1, wherein the deployment member is made from a thermally transmissive material.

24. The medical device according to claim 1, wherein the deployment member is comprised of at least one inflatable element.

25. The medical device according to claim 1, wherein the deployment member is made from a shape memory material.

26. The medical device according to claim 1, wherein the deployment member includes a spring.

27. The medical device according to claim 1, wherein the deployment member has a rigidity that can be modified by changes in temperature.

28. A medical device for thermally affecting tissue, comprising:
a heat exchanger defining a chamber, an input lumen and an output lumen, wherein the chamber is adapted to receive a first fluid, with the heat exchanger being deformable upon contact with tissue, and
a deployment member secured to at least a portion of the heat exchanger, wherein the deployment member is adapted to receive a second fluid, wherein the second fluid is pressurized to obtain a desired rigidity of the deployment member.

29. A method for thermally affecting tissue, comprising the steps of:
positioning a medical device in proximity to an area of tissue, the medical device being comprised of a heat exchanger and a deployment member secured to at least a portion of the heat exchanger,
introducing an inflation fluid into the deployment member,
positioning the medical device into thermal communication with a tissue,
circulating a thermally transmissive fluid through the heat exchanger, and
allowing the medical device to thermally affect the tissue.

30. The method according to claim 29, further comprising the step of evacuating the thermally transmissive fluid from the heat exchanger.

31. The method according to claim 30, further comprising the steps of evacuating the inflation fluid from the deployment member and removing the device from thermal communication with the tissue.

32. The method according to claim 29, wherein the tissue is brain tissue.
